(19) United States
(12) Patent Application Publication
(10) Pub. No.: US 2017/0143854 A1
(43) Pub. Date: May 25, 2017

(54) NANOPARTICLES AND METHODS OF MAKING

(71) Applicant: Massachusetts Institute of Technology, Cambridge, MA (US)

(72) Inventors: Ritchie Chen, Cambridge, MA (US); Polina Olegovna Anikeeva, Somerville, MA (US); Alexandra Sourakov, Boston, MA (US)

(73) Assignee: Massachusetts Institute of Technology, Cambridge, MA (US)

(21) Appl. No.: 15/359,606

(22) Filed: Nov. 22, 2016

Related U.S. Application Data
(60) Provisional application No. 62/259,036, filed on Nov. 23, 2015.

(51) Int. Cl. A61K 49/18

(52) U.S. Cl. CPC A61K 49/1818

(57) ABSTRACT

Magnetic nanoparticles and synthesis of synthesis are described.

### CLAIM OF PRIORITY

This application claims the benefit of prior U.S. Provisional Application No. 62/259,036 filed on Nov. 23, 2015, which is incorporated by reference in its entirety.

### STATEMENT REGARDING FEDERALLY SPONSORED RESEARCH OR DEVELOPMENT

This invention was made with Government support under Grant Nos. DMR-1419807 and DMR-0819762 awarded by the National Science Foundation and under Grant No. D13AP00045 awarded by the U.S. Department of Interior. The Government has certain rights in the invention.

### FIELD OF THE INVENTION

The invention relates to nanoparticles and methods of making.

### BACKGROUND

From magnetic resonance imaging to cancer hyperthermia and wireless control of cell signaling, iron oxide nanoparticles produced by thermal decomposition methods are ubiquitous across biomedical applications.

### SUMMARY

From magnetic resonance imaging to cancer hyperthermia and wireless control of cell signaling, iron oxide nanoparticles produced by thermal decomposition methods are ubiquitous across biomedical applications. While well-established synthetic protocols allow for precise control over the size and shape of the magnetic nanoparticles, structural defects within seemingly single-crystalline materials contribute to variability in the reported magnetic properties. In general, stabilization of metastable wüstite in commonly used hydrocarbon solvents contributed to significant cation disorder, leading to nanoparticles with poor hyperthermic efficiencies and transverse relaxivities. By introducing aromatic ethers that undergo radical decomposition upon thermolysis, the electrochemical potential of the solvent environment was tuned to favor the ferrimagnetic phase. Structural and magnetic characterization identified hallmark features of nearly defect-free ferrite nanoparticles that could not be demonstrated through post-synthesis oxidation, with nearly 500% improvement in the specific loss powers and transverse relaxivity times compared to similarly sized nanoparticles containing defects. The improved crystallinity of the nanoparticles enabled rapid wireless control of intracellular calcium. Surprisingly, redox tuning during solvent thermolysis can generate potent theranostic agents through selective phase control.

In one aspect, a method of preparing a redox-active nanoparticle includes selecting a solvent to optimize redox tuning of a reaction medium, and decomposing a precursor compound at a reflux temperature of the solvent to produce the nanoparticle.

In another aspect, a magnetic nanoparticle population can include a plurality monodisperse ferrite particles having a size of between 7 and 30 nm.

In another aspect, a method of imaging can include introducing the nanoparticle population into a subject; and creating a magnetic particle imaging signal of the subject.

In certain circumstances, the oxidized nanoparticle includes iron, manganese, cobalt, nickel or copper, or binary or ternary mixtures thereof.

In certain circumstances, the method can include oxidizing the nanoparticle.

In certain circumstances, the oxidized nanoparticle can be an inverse spinel phase iron oxide.

In certain circumstances, the solvent can include dibenzyl ether, dibenzyl ether, diphenyl ether, anisole, phenetole, an aromatic ester, an aromatic acetal, an aromatic aminal or an aromatic anhydride.

In certain circumstances, the solvent can be a mixture. For example, the mixture can include a high boiling point alkene and a high boiling point ether. The high boiling point can be at least 280° C., for example, between 285° C. and 325° C.

In certain circumstances, the solvent can include a mixture of two or more of octadecane, 1-octadecene (ODE), squalene (SQE), dioctyl ether, and dibenzyl ether (DBE).

In certain circumstances, the compound can include an iron oleate.

In certain circumstances, the nanoparticle can include ferrite.

In certain circumstances, the nanoparticle can have a size of between 7 and 30 nm.

Other aspects, embodiments, and features will be apparent from the following description, the drawings, and the claims.

### BRIEF DESCRIPTION OF THE DRAWINGS

FIGS. 1A-1K represent solvent influences phases found in iron oxide nanoparticles. Transmission electron micrographs of ferrite nanoparticles synthesized in (A) squalene and (B) dibenzyl ether. (C) Powder x-ray diffractograms of nanoparticles synthesized in various solvents. Red asterix denotes prominent wüstite peaks found in all syntheses except for iron oleate decomposition in dibenzyl ether. Reference pattern is given for magnetite (black) and wüstite (red). (D) High-resolution transmission electron micrographs of individual particles synthesized in squalene and in (E) dibenzyl ether. (F, G) Fast Fourier transform of individual particles. Reconstructed images filtered from specific plane orientations for (H, I) {311} magnetite and (J,K) {111} wüstite.

FIGS. 2A-2H represent solvent redox mechanism of ferrimagnetic nanoparticles with tunable size. (A) FTIR absorbance spectroscopy of pure reactants with the aldehyde of benzaldehyde (grey *) and vinyl double bond of ODE (red *) starred. Evolution of reaction at 200° C., at reflux, and at 1 hr of refluxing for FeOl₃ decomposed in (B) ODE, (C) DBE, and (D) 2:1 volume ratio of ODE:DBE. The absorption peak positions of the double bond of ODE (red dashed line) and of the aldehyde of benzaldehyde (grey solid line) are marked. (E) Reaction space as a function of volume percentage of ODE in a co-solvent of ODE and DBE and oleic acid (OAc):FeOl₃ ratio. Nanoparticle size <15 nm in diameter are denoted as □ and for diameters >15 nm. Optimal size range (15-30 nm) of ferrimagnetic nanoparticles is circled with the red dashed line. Transmission electron microscopy images of nanoparticles synthesized in a 2:1 volume ratio of ODE to DBE with an OAc:FeOl₃ molar ratio of 1:1, 2:1, and 3:1 resulting in particle diameters of (F) 15±1.5 nm, (G) 19±1.8 nm, and (H) 27±1.9 nm.

FIGS. 3A-3F represent structural and magnetic characterization of nearly defect-free nanoparticles. (A) Schematic illustrating the four different phase compositions synthesized. Pyrolysis in SQE leads to biphasic nanoparticles which can be oxidized with TMAO into strained single-phase Fe_{3-δ}O₄. Truncated icosahedrons composed of Fe_{3-δ}O₄ are synthesized by SORT and can be oxidized to γ-Fe₂O₃. OAc=oleic acid. (B) TEM images of similarly sized ˜25 nm nanoparticles corresponding to the four chemical treatments (SQE=biphasic FeO/Fe_{3-δ}O₄, SQE-oxidized=strained Fe_{3-δ}O₄, 2:1 SQE:DBE (SORT)=Fe_{3-δ}O₄, 2:1 SQE:DBE (SORT Ox)=γFe₂O₃). Scale bar=50 nm. (C) Full-width at half max values of the (220) (orange), (400) (blue), and (440) (grey) diffraction peaks of the four samples. (D) Field-cooled magnetization curve measured at 5K for the four samples (SQE (pink), SQE Ox (black), SORT (red), SORT Ox (gray)). (E) Measured exchange-bias (μₒH_{EB}) and coercive field (μₒH_{C}) for the four samples calculated from the magnetization curve. (inset) low-field region of the magnetization curve in D. (F) Magnetization curve as a function of temperature for SORT (red) and SQE Ox (black) nanoparticles. Neél temperature (T_{N}) and the Verwey transition temperature (Tᵥ) are marked with the grey dashed line.

FIGS. 4A-4E represent water-soluble PEG-coated nanoparticles with high transverse relaxivities and specific loss powers (SLP). (A) Number distribution of the hydrodynamic diameter of 25 nm SORT nanoparticles dispersed in water and in physiological buffer (Tyrode's solution) measured by dynamic light scattering. (inset) TEM image of PEG-coated nanoparticles. Zeta-potential measurements (ξ) indicate a charge neutral surface. (B) T₂-weighted magnetic resonance images at 7 T of iron oxide nanoparticles at different concentrations for 25 nm nanoparticles from the four synthetic protocols. (C) Comparison of the r₂ values measured. SLP as a function of field amplitude Hₒ at f=100 kHz for (D) ˜25 nm nanoparticles composed of different phases and (E) nanoparticles with different diameters synthesized by SORT.

FIGS. 5A-5C represent high-performance nanoparticles enables rapid magnetothermal control of intracellular concentration. (A) Normalized fluorescence traces of gCaMP6s as a function of time averaged over 50 TRPV1⁺ HEK293FT cells in response to an applied field (duration indicated by AMF ON, f=150 kHz, Hₒ=30 kA/m) for SORT (red line) and SQE Oxidized (black line) nanoparticles at a concentration of 2 mg/mL [Fe]. The shaded grey area indicates standard error. (Inset) Temperature profile of SORT (red) and SQE Oxidized (black) nanoparticle solutions during cell stimulation. Heat maps of fluorescence intensity normalized to baseline before and during field stimulus for HEK293FT cells incubated with (B) SORT and (C) SQE Oxidized nanoparticles. Cells with observable minor onset are denoted with an *. Scale bar=100 μm.

FIG. 6, panels A-E, are TEM micrographs of ferrite nanoparticles synthesized from Fe(Ol)₃ (A-C) and Fe(acac)₃ (D,E) decomposition in the indicated solvent. Biphasic core-shell nanoparticles are evident based on contrast. White arrow indicates core-shell geometry even in dioctyl ether.

FIG. 7 represents room temperature magnetization curves for the various ferrite nanoparticles synthesized in different solvents: dioctyl ether (DOE), dibenzyl ether (DBE), octadecane (ODA), 1-octadecene (ODE), and squalene (SQE)

FIG. 8 represents integrated area of the vinyl peak of ODE (═C—H, 910 cm⁻¹) after 30 minutes of reflux normalized to the initial integrated area of the same peak at 200 degrees C. for different volume percentages of ODE:DBE with all other reaction parameters remaining the same.

FIG. 9 represents a TEM image of nanoparticles synthesized in 15 mL of ODE with 10 mmol oleic acid and 10 mmol of benzaldehyde. Without the addition of DBE, the nanoparticles have core-shell morphology characteristic of biphasic nanoparticles, demonstrating that radicals generated during decomposition of DBE into benzaldehyde is necessary for the production of nearly-defect free nanoparticles.

FIGS. 10A-10C represent powder x-ray diffractogram of (A) SQE biphasic nanoparticles (red) and SQE oxidized nanoparticles containing defects (grey), and (B) as-synthesized ferrite nanoparticles that underwent SORT (black) and SORT oxidized nanoparticles (pink). (C) Reference pattern of wüstite (FeO), magnetite (Fe₃O₄), and maghemite (γ-Fe₂O₃).

FIG. 11 represents images of: (A) High-resolution transmission micrograph of single nanoparticle synthesized by SORT. (B) Fast Fourier transform of the single particle image with the {111} and {220} marked ∘ and □ respectively. (D) Reconstructed image exhibiting single-crystalline, inverse spinel nanoparticles free of the wüstite phase.

FIG. 12 represents fluorescence image of HEK293FT cells co-transfected with the heat-sensitive ion channel TRPV1 (upper) and gCaMP6s (lower).

FIG. 13 is a close-up of FIG. 5A, showing minor onset within ˜750 ms of applied field (AMF ON) for SORT synthesized nanoparticles.

### DETAILED DESCRIPTION

Ferrite-based magnetic nanoparticles (MNPs) synthesized from the thermal decomposition of organometallic precursors exhibit some of the highest hyperthermic efficiencies and magnetic resonance (MR) transverse relaxivities measured to date. See, for example, Noh, S.; Na, W.; Jang, J.; Lee, J.; Lee, E. J.; Moon, S. H.; Lim, Y.; Shin, J.; Cheon, J. Nano Letters 2012, 12, (7), 3716-3721; Guardia, P.; Di Corato, R.; Lartigue, L.; Wilhelm, C.; Espinosa, A.; Garcia-Hernandez, M.; Gazeau, F.; Manna, L.; Pellegrino, T. ACS Nano 2012, 6, (4), 3080-3091; Jang, J.; Nah, H.; Lee, J.; Moon, S. H.; Kim, M. G.; Cheon, J. Angewandte Chemie, International Edition 2009, 48, (7), 1234-1238; and Lee, N.; Choi, Y.; Lee, Y.; Park, M.; Moon, W. K.; Choi, S. H.; Hyeon, T. Nano Letters 2012, 12, (6), 3127-3131, each of which is incorporated by reference in its entirety. These application-specific performance metrics depend on the nanoparticle's magnetic properties and are determined by its crystal structure and composition. Surprisingly, despite an abundance of protocols detailing the production of ferrite nanoparticles, phase control over the various iron oxide polymorphs remains a synthetic challenge due to the local stabilization of thermodynamically unstable phases at nanoscale interfaces. See, for example, van Embden, J.; Chesman, A. S. R.; Jasieniak, J. J. Chemistry of Materials 2015, 27, (7), 2246-2285; Navrotsky, A.; Mazeina, L.; Majzlan, J. Science 2008, 319, (5870), 1635-1638; Redl, F. X.; Black, C. T.; Papaefthymiou, G. C.; Sandstrom, R. L.; Yin, M.; Zeng, H.; Murray, C. B.; O'Brien, S. P. Journal of the American Chemical Society 2004, 126, (44), 14583-14599; Wetterskog, E.; Tai, C.-W.; Grins, J.; Bergström, L.; Salazar-Alvarez, G. ACS Nano 2013, 7, (8), 7132-7144, each of which is incorporated by reference in its entirety. Depending on the oxidation state, iron oxide can exist in three magnetic phases: fully oxidized maghemite (γ-Fe₂O₃), mixed valent Fe^{2+/3+} magnetite (Fe₃O₄), and reduced metastable wüstite (FeOₓO, x=0.83-0.96). See, for example, Wetterskog, E.; Tai, C.-W.; Grins, J.; Bergström, L.; Salazar-Alvarez, G. ACS Nano 2013, 7, (8), 7132-7144; O'Brien, S.; Brus, L.; Murray, C. B. Journal of the American Chemical Society 2001, 123, (48), 12085-12086, each of which is incorporated by reference in its entirety. While magnetite and maghemite adopt an inverse spinel ferrimagnetic (FiM) configuration, wüstite is weakly paramagnetic at room temperature and antiferromagnetic (AFM) below its Neél temperature with a rock-salt structure that is thermodynamically stable only above 560° C. See, for example, Redl, F. X.; Black, C. T.; Papaefthymiou, G. C.; Sandstrom, R. L.; Yin, M.; Zeng, H.; Murray, C. B.; O'Brien, S. P. Journal of the American Chemical Society 2004, 126, (44), 14583-14599, which is incorporated by reference in its entirety. Because all three crystal structures possess a face-centered cubic oxygen sublattice with the phase difference determined only by the coordination state of the iron ions, cation disorder may emerge during nanoparticle nucleation and growth. See, for example, Wetterskog, E.; Tai, C.-W.; Grins, J.; Bergström, L.; Salazar-Alvarez, G. ACS Nano 2013, 7, (8), 7132-7144; O'Brien, S.; Brus, L.; Murray, C. B. Journal of the American Chemical Society 2001, 123, (48), 12085-12086; Levy, M.; Quarta, A.; Espinosa, A.; Figuerola, A.; Wilhelm, C.; Garcia-Hernandez, M.; Genovese, A.; Falqui, A.; Alloyeau, D.; Buonsanti, R.; Cozzoli, P. D.; Garcia, M. A.; Gazeau, F.; Pellegrino, T. Chemistry of Materials 2011, 23, (18), 4170-4180; Walter, A.; Billotey, C.; Garofalo, A.; Ulhaq-Bouillet, C.; Lefèvre, C.; Taleb, J.; Laurent, S.; Vander Elst, L.; Muller, R. N.; Lartigue, L.; Gazeau, F.; Felder-Flesch, D.; Begin-Colin, S. Chemistry of Materials 2014, 26, (18), 5252-5264, each of which is incorporated by reference in its entirety. Resulting phase impurities and defects lead to low hysteretic power losses and transverse relaxivities because of unfavorable exchange interactions between the AFM and FiM phases. See, for example, Levy, M.; Quarta, A.; Espinosa, A.; Figuerola, A.; Wilhelm, C.; Garcia-Hernandez, M.; Genovese, A.; Falqui, A.; Alloyeau, D.; Buonsanti, R.; Cozzoli, P. D.; Garcia, M. A.; Gazeau, F.; Pellegrino, T. Chemistry of Materials 2011, 23, (18), 4170-4180; Walter, A.; Billotey, C.; Garofalo, A.; Ulhaq-Bouillet, C.; Lefèvre, C.; Taleb, J.; Laurent, S.; Vander Elst, L.; Muller, R. N.; Lartigue, L.; Gazeau, F.; Felder-Flesch, D.; Begin-Colin, S. Chemistry of Materials 2014, 26, (18), 5252-5264, each of which is incorporated by reference in its entirety.

Pyrolysis of inexpensive and environmentally benign iron acetylacetonate (Fe(acac)₃) and iron oleate (FeOl₃) precursors is widely used to produce ferrite nanoparticles due to scalability, and tunability in chemical composition, size, and shape. See, for example, Yu, W. W.; Falkner, J. C.; Yavuz, C. T.; Colvin, V. L. Chemical Communications 2004, (20), 2306-2307; Park, J.; An, K.; Hwang, Y.; Park, J.; Noh, H.; Kim, J.; Park, J.; Hwang, N.; Hyeon, T. Nat Mater 2004, 3, (12), 891-895; Bao, N.; Shen, L.; Wang, Y.; Padhan, P.; Gupta, A. Journal of the American Chemical Society 2007, 129, (41), 12374-12375; Bronstein, L. M.; Huang, X.; Retrum, J.; Schmucker, A.; Pink, M.; Stein, B. D.; Dragnea, B. Chemistry of Materials 2007, 19, (15), 3624-3632, each of which is incorporated by reference in its entirety. Inconsistencies in the magnetic properties of the as-synthesized nanoparticles, however, highlight the challenges in controlling the different magnetic polymorphs of iron oxide. See, for example, Hai, H. T.; Yang, H. T.; Kura, H.; Hasegawa, D.; Ogata, Y.; Takahashi, M.; Ogawa, T. Journal of Colloid and Interface Science 2010, 346, (1), 37-42; Bao, N.; Shen, L.; Wang, Y.; Padhan, P.; Gupta, A. Journal of the American Chemical Society 2007, 129, (41), 12374-12375; Bronstein, L. M.; Huang, X.; Retrum, J.; Schmucker, A.; Pink, M.; Stein, B. D.; Dragnea, B. Chemistry of Materials 2007, 19, (15), 3624-3632; Kovalenko, M. V.; Bodnarchuk, M. I.; Lechner, R. T.; Hesser, G.; Schäffler, F.; Heiss, W. Journal of the American Chemical Society 2007, 129, (20), 6352-6353; Shavel, A.; Liz-Marzan, L. M. Physical Chemistry Chemical Physics 2009, 11, (19), 3762-3766; Pichon, B. P.; Gerber, O.; Lefevre, C.; Florea, I.; Fleutot, S.; Baaziz, W.; Pauly, M.; Ohlmann, M.; Ulhaq, C.; Ersen, O.; Pierron-Bohnes, V.; Panissod, P.; Drillon, M.; Begin-Colin, S. Chemistry of Materials 2011, 23, (11), 2886-2900, each of which is incorporated by reference in its entirety. Typically, Fe(acac)₃ decomposition produces non-stoichiometric magnetite (Fe_{3-δ}O₄) with high saturation magnetization (Mₛ) approaching values of the bulk material (94 A·m²/kg), (see, for example, Sun, S.; Zeng, H.; Robinson, D. B.; Raoux, S.; Rice, P. M.; Wang, S. X.; Li, G. Journal of the American Chemical Society 2003, 126, (1), 273-279; Kim, D.; Lee, N.; Park, M.; Kim, B. H.; An, K.; Hyeon, T. Journal of the American Chemical Society 2008, 131, (2), 454-455, each of which is incorporated by reference in its entirety) while synthesis from FeOl₃ frequently results in biphasic nanoparticles composed of an AFM core and a FiM shell. See, for example, Bronstein, L. M.; Huang, X.; Retrum, J.; Schmucker, A.; Pink, M.; Stein, B. D.; Dragnea, B. Chemistry of Materials 2007, 19, (15), 3624-3632; Kovalenko, M. V.; Bodnarchuk, M. I.; Lechner, R. T.; Hesser, G.; Schäffler, F.; Heiss, W. Journal of the American Chemical Society 2007, 129, (20), 6352-6353; Shavel, A.; Liz-Marzan, L. M. Physical Chemistry Chemical Physics 2009, 11, (19), 3762-3766; Pichon, B. P.; Gerber, O.; Lefevre, C.; Florea, I.; Fleutot, S.; Baaziz, W.; Pauly, M.; Ohlmann, M.; Ulhaq, C.; Ersen, O.; Pierron-Bohnes, V.; Panissod, P.; Drillon, M.; Begin-Colin, S. Chemistry of Materials 2011, 23, (11), 2886-2900, each of which is incorporated by reference in its entirety. Antiphase boundaries that form at the subdomain interfaces in this AFM/FiM coupled system lead to undesirable magnetic properties, including low Mₛ, high-field susceptibility, and exchange bias. See, for example, Wetterskog, E.; Tai, C.-W.; Grins, J.; Bergström, L.; Salazar-Alvarez, G. ACS Nano 2013, 7, (8), 7132-7144, which is incorporated by reference in its entirety. Although post-synthesis oxidation has been proposed to yield only the FiM phase, (see, for example, Casula, M. F.; Jun, Y.-w.; Zaziski, D. J.; Chan, E. M.; Corrias, A.; Alivisatos, A. P. Journal of the American Chemical Society 2006, 128, (5), 1675-1682; Sun, X.; Frey Huls, N.; Sigdel, A.; Sun, S. Nano Letters 2012, 12, (1), 246-251; Hai, H. T.; Kura, H.; Takahashi, M.; Ogawa, T. Journal of Applied Physics 2010, 107, (9), 09E301-09E301-3; Chen, R.; Christiansen, M. G.; Anikeeva, P. ACS Nano 2013, 7, (10), 8990-9000, each of which is incorporated by reference in its entirety) defects may persist due to limited diffusion in disordered cationic layers. See, for example, Wetterskog, E.; Tai, C.-W.; Grins, J.; Bergström, L.; Salazar-Alvarez, G. ACS Nano 2013, 7, (8), 7132-7144, which is incorporated by reference in its entirety. Identifying general rules to tune the compositional range of iron oxide is therefore crucial for optimizing the magnetic properties of the MNPs.

In this study, it was found that in addition to the precursor and the surfactant, the solvent played an equally vital role in defining the phase purity, size, and shape of the as-synthesized ferrite nanoparticles. By contrasting well-established protocols, it was observed that Fe(acac)₃ pyrolysis in dibenzyl ether (290° C.) yielded single-crystalline nanoparticles with bulk-like magnetic properties, while higher boiling point (>300° C.) alkene hydrocarbons, required to fully decompose the FeOl₃ precursor, produced biphasic nanoparticles. It was found that this large difference in phase purity was largely determined by the solvent's redox activity to control the valence state of iron. Thermolysis of aromatic ethers produced oxidizing species that stabilized the inverse spinel phase, while alkene hydrocarbons had reducing effects that favored the formation of wüstite. Controlling this nonaqueous redox environment enabled reproducible and scalable synthesis of nearly defect-free FiM MNPs in the 10-30 nm range without the need for post-synthesis modification. These MNPs exhibited increased transverse relaxivities and enhanced hyperthermic performance in comparison to similarly sized nanoparticles subjected to conventional post-synthesis oxidation methods. Following phase transfer into physiological media, these MNPs enabled rapid wireless magnetothermal control of intracellular calcium with sub-second latency.

Nanoparticles can be prepared through the thermal decomposition of metal-complex precursors in hot non-hydrolytic organic solution containing surfactants. Thermal decomposition of the precursors can generate monomers and their aggregation above a supersaturation level can induce nucleation and subsequent nanoparticle growth. During these stages, it is possible to control the size, composition, and magneto-crystalline phase of nanoparticles by tuning growth parameters, such as monomer concentration, crystalline phase of the nuclei, choice of solvent and surfactants, growth temperature and time, and surface energy. Metal ferrite nanoparticles can be synthesized from precursors such as iron pentacarbonyl, iron cupferron, iron tris(2,4-pentadionate), and iron fatty acid complexes, in hot organic solvents containing fatty acids and amine surfactants. Nanoparticle size can be tuned within the range of 1 nm to approximately 150 nm.

The nanoparticles have a size between 1 and 100 nanometers, between 5 and 50 nanometers, or between 7 and 30 nanometers. The resulting nanoparticles can form a monodisperse population, meaning that the size or dimension (i.e., diameter) of the nanoparticles is have a dimension or size that varies by less than 10%, i.e., +/−10%.

Thus, the method of preparing a redox-active nanoparticle includes selecting a solvent to optimize redox tuning of a reaction medium. As noted above, this is essential to allow the proper growth of the nanoparticles. The decomposition of a precursor compound at a reflux temperature of the solvent produces the nanoparticle.

The selection of the solvent can be made to optimize the formation of the nanoparticle. When the solvent includes dibenzyl ether, formation of ferrite nanoparticles having controllable size is achieved. The solvent can be a mixture of solvents, which can include dibenzyl ether, or other high boiling point aromatic ethers such as diphenyl ether, anisole, phenetole, and aromatic esters, as well as acetals, aminals and anhydrides which can undergo free radical decomposition, where the aromatic ring can stabilize the oxidizing radical, can similarly be used in solvent redox. In particular, the solvent can include a mixture of two or more of octadecane, 1-octadecene (ODE), squalene (SQE), dioctyl ether, and dibenzyl ether (DBE). Examples of solvent combinations and resulting magnetic nanoparticle phase are summarized in Table 1.

TABLE 1

| Solvent 1 | Solvent 2 | Resulting magnetic phase |
|---|---|---|
| 1-octadecene | Dibenzyl ether | Inverse spinel |
| squalene | Dibenzyl ether | Inverse spinel |
| 1-octadecene | Diphenyl ether | Inverse spinel |
| Trioctylamine | Dibenzyl ether | Inverse spinel |
| 1-eicosene | Dibenzyl ether | Inverse spinel |
| 1-octadecene | Dioctyl ether | Inverse spinel/wustite |
| 1-octadecene | none | Inverse spinel/wustite |
| 1-eicosene | none | Inverse spinel/wustite |
| Dioctyl ether | none | Inverse spinel/wustite |
| Squalene | none | Inverse spinel/wustite |
| Dibenzyl ether | None | Inverse spinel |

The oxidized nanoparticle can include iron, manganese, cobalt, nickel or copper, or binary or ternary mixtures thereof. For example, beyond iron oleate, binary and ternary oleate mixtures comprised of other transition metals can result in modification to the resulting magnetic properties in the nanoparticles. For example iron-zinc oleate, where the zinc in the Fe/Zn mixture can be varied from 1-50%, can boost the saturation magnetization of the nanoparticle, enhancing its biomedical performance as T2 contrast agents and for hyperthermia. Iron-cobalt and Iron-manganese can be similarly incorporated to modify the magnetic properties.

In addition, manganese-oleate, cobalt-oleate, nickel-oleate, copper-oleate can be used to generate other transition metal oxide nanoparticles using solvent redox to influence the resulting redox phase, which is useful for catalysis and as battery materials and other electrochemical devices. A method of imaging can include introducing the nanoparticle population into a subject; and creating a magnetic particle imaging signal of the subject.

### Results and Discussion

Solvent Choice Dictates Iron Polymorphs in MNPs.

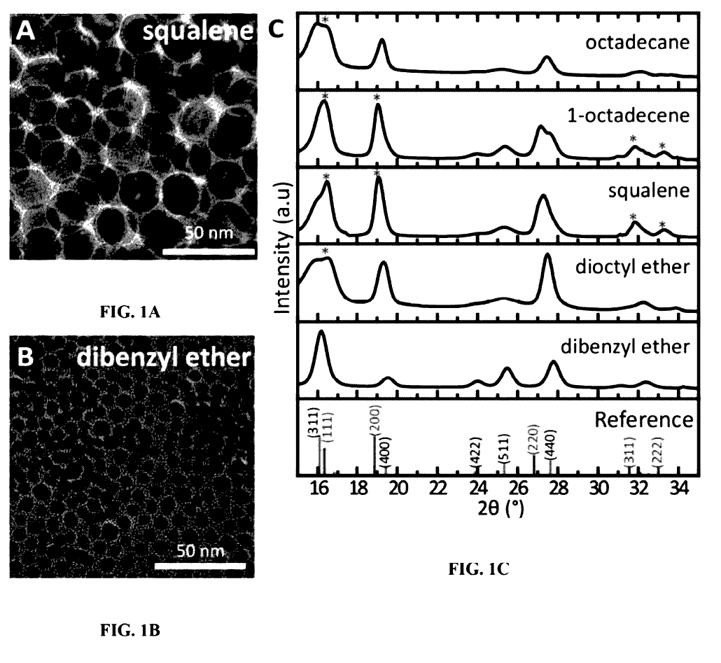

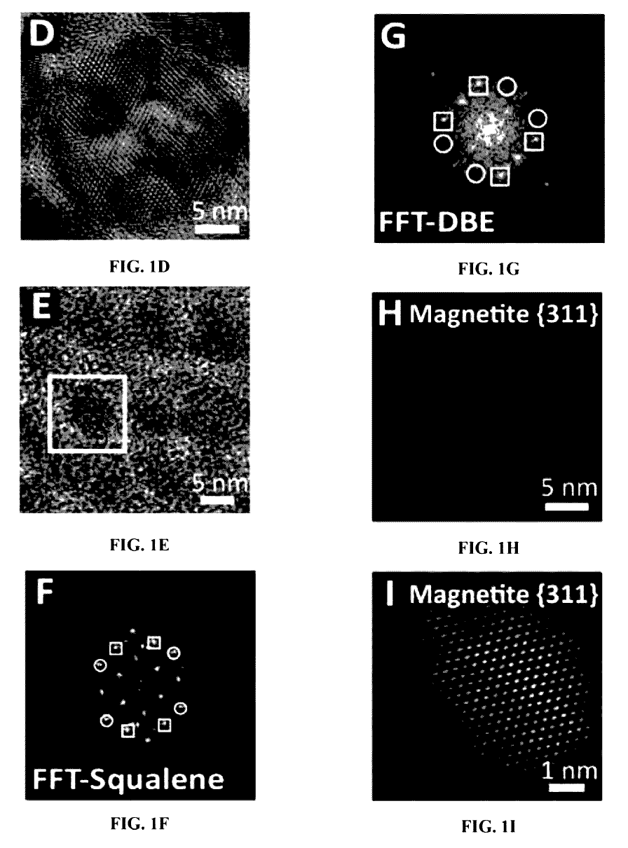

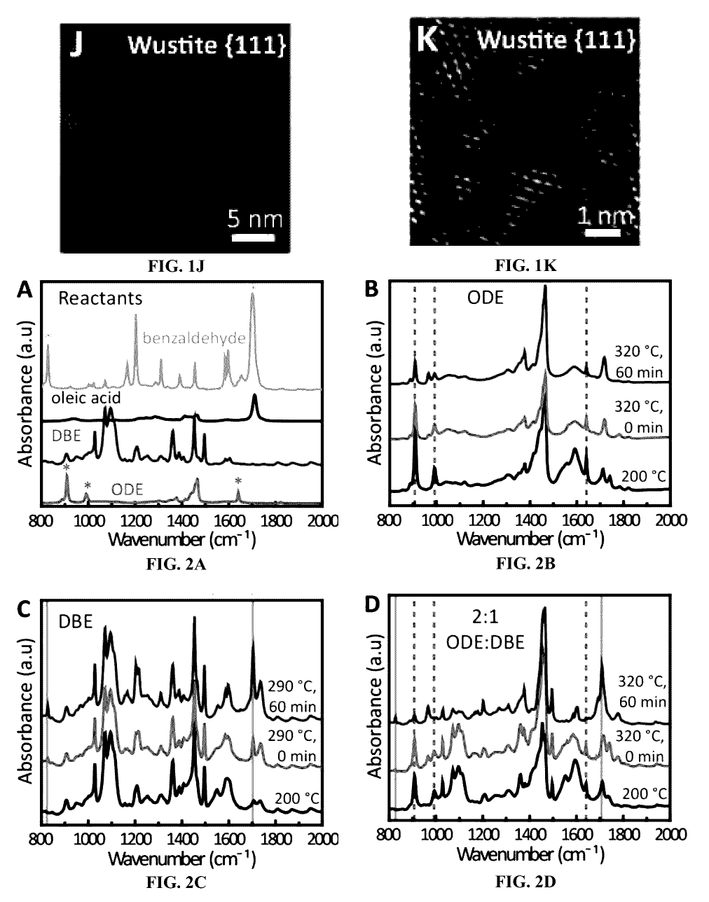

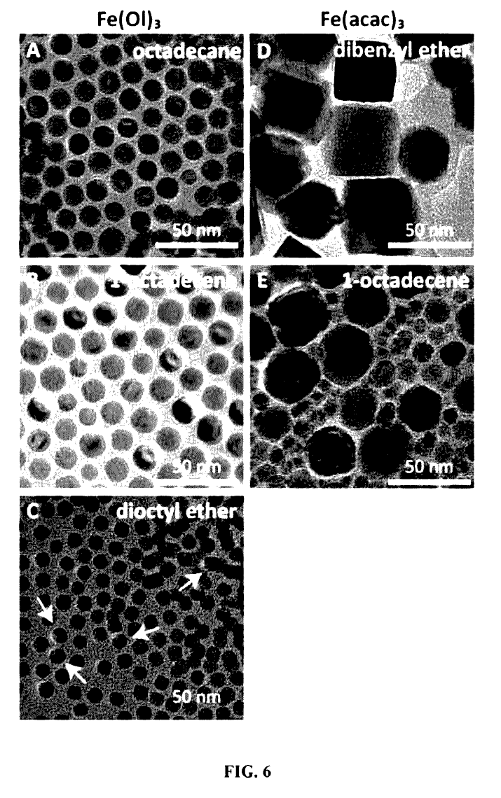

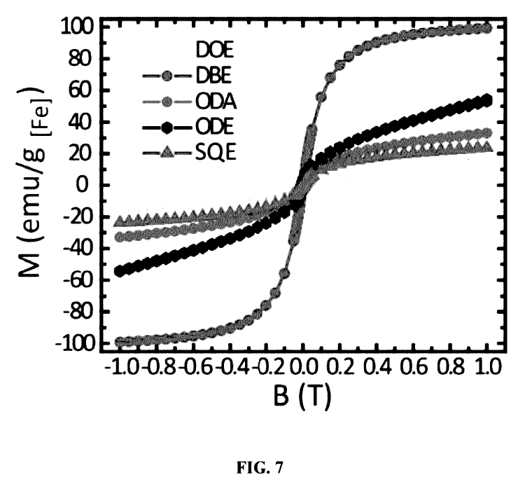

To assess the influence of solvent on crystal structure, monodisperse iron oxide nanoparticles were synthesized by FeOl₃ decomposition in octadecane, 1-octadecene (ODE), squalene (SQE), dioctyl ether, and dibenzyl ether (DBE). Heterogeneous contrast from high-resolution transmission electron (HRTEM) micrographs revealed core-shell architecture for nanoparticles produced in all solvents except in DBE (FIGS. 1A and 1B, FIG. 6, panels A-C). Fe(acac)₃ pyrolysis similarly produced core-shell nanoparticles in ODE but not in DBE with broader size distribution (FIG. 6, panels D, E). In comparison to Fe(acac)₃, which begins to decompose at ˜180° C., FeOl₃ enabled finer control over the nanoparticle size and size distribution due to the proximity of its decomposition temperature (>300° C.) to the boiling points of the chosen solvents. See, for example, van Embden, J.; Chesman, A. S. R.; Jasieniak, J. J. Chemistry of Materials 2015, 27, (7), 2246-2285, which is incorporated by reference in its entirety. Bulk powder x-ray diffraction (XRD) indicated that pyrolysis in octadecane, ODE, SQE, and dioctyl ether produced nanoparticles containing both rock-salt and inverse spinel phases, while decomposition in DBE yielded only inverse spinel nanoparticles (FIG. 1C). The phases of an individual nanoparticle synthesized in SQE were mapped using fast Fourier transform (FFT), which revealed a rock-salt core and an inverse spinel shell (FIGS. 1D, 1F, 1H and 1J). Although the diffraction peaks corresponding to the {111} and {311} planes from wüstite and magnetite overlap due to similar interplanar spacing, the plane angle relative to the <200> direction is unique and allows for identification of the two phases as denoted by the spots marked with circles ∘ ({111} wüstite, d=0.2498 nm) and squares □ ({311} magnetite, d=0.2534 nm) in FIG. 1F. Reconstructed images filtered for the {111} reflections of wüstite showed preferential distribution within the core, while the inverse FFT from the {311} planes assigned the inverse spinel phase predominately to the shell. This morphology is consistent with an oxidation mechanism that converts metastable wüstite into magnetite at the MNP surface (FIGS. 1H and 1J). See, for example, Pichon, B. P.; Gerber, O.; Lefevre, C.; Florea, I.; Fleutot, S.; Baaziz, W.; Pauly, M.; Ohlmann, M.; Ulhaq, C.; Ersen, O.; Pierron-Bohnes, V.; Panissod, P.; Drillon, M.; Begin-Colin, S. Chemistry of Materials 2011, 23, (11), 2886-2900, which is incorporated by reference in its entirety. By contrast, single-crystalline inverse spinel 6.7±0.7 nm nanoparticles were synthesized in DBE (FIGS. 1E, 1G, 1I and 1K). Room-temperature vibrating sample magnetometry (VSM) showed high-field susceptibility and low Mₛ for all MNPs with the exception of those synthesized in DBE, which is characteristic of ferrite systems with strained AFM/FiM interfaces (FIG. 7). See, for example, Wetterskog, E.; Tai, C.-W.; Grins, J.; Bergström, L.; Salazar-Alvarez, G. ACS Nano 2013, 7, (8), 7132-7144; Chen, R.; Christiansen, M. G.; Anikeeva, P. ACS Nano 2013, 7, (10), 8990-9000, each of which is incorporated by reference in its entirety.

Redox Active Species are Generated During Solvent Thermolysis.

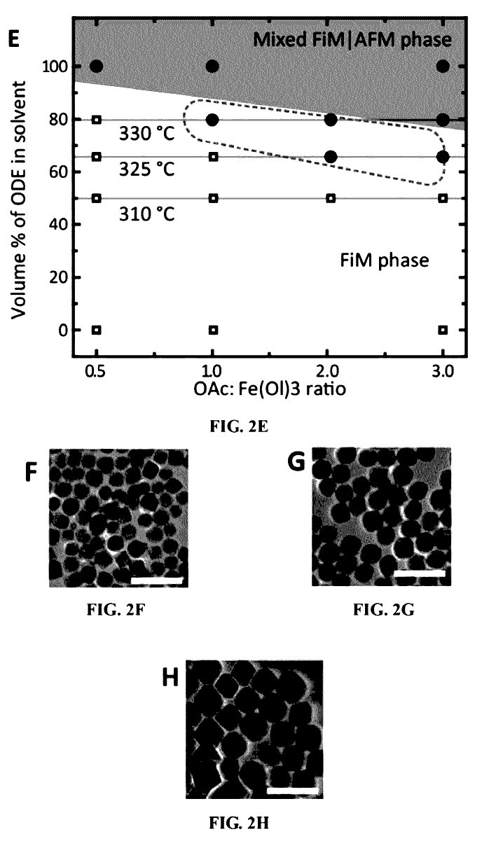

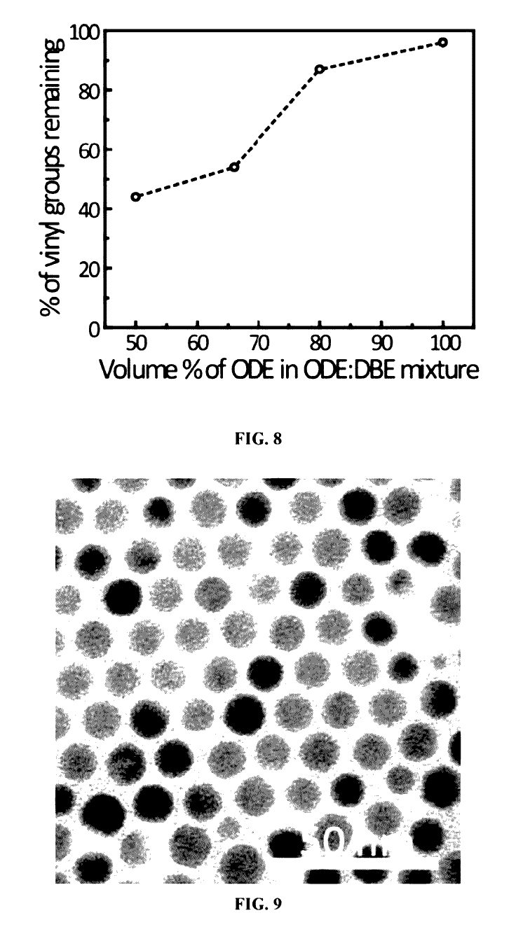

The solvents ODE and SQE were selected to examine the reductive tendencies of unsaturated bonds in alkene hydrocarbons. See, for example, Shavel, A.; Liz-Marzan, L. M. Physical Chemistry Chemical Physics 2009, 11, (19), 3762-3766; Chen, 0.; Chen, X.; Yang, Y.; Lynch, J.; Wu, H.; Zhuang, J.; Cao, Y. C. Angewandte Chemie International Edition 2008, 47, (45), 8638-8641, each of which is incorporated by reference in its entirety. The aromatic ether DBE was investigated for potential oxidative effects, since its decomposition can generate intermediate radical products. See, for example, Gilbert, K. E.; Gajewski, J. J. The Journal of Organic Chemistry 1982, 47, (25), 4899-4902, which is incorporated by reference in its entirety. While the valence state of iron exists only in the 3+ state in the precursor, (see, for example, Bao, N.; Shen, L.; Wang, Y.; Padhan, P.; Gupta, A. Journal of the American Chemical Society 2007, 129, (41), 12374-12375, which is incorporated by reference in its entirety), production of CO₂ during FeOl₃ decomposition was reported to be sufficient to reduce Fe³⁺ to Fe²⁺. See, for example, Kwon, S. G.; Piao, Y.; Park, J.; Angappane, S.; Jo, Y.; Hwang, N.-M.; Park, J.-G.; Hyeon, T. Journal of the American Chemical Society 2007, 129, (41), 12571-12584, which is incorporated by reference in its entirety. Recent studies have also demonstrated that the moles of CO₂ emitted over the course of a reaction exceeded the moles of reactants by an order of magnitude, indicating that oxidation of ODE, a commonly used solvent, into CO₂ may contribute to the reduction of Fe³⁺. See, for example, Hai, H. T.; Yang, H. T.; Kura, H.; Hasegawa, D.; Ogata, Y.; Takahashi, M.; Ogawa, T. Journal of Colloid and Interface Science 2010, 346, (1), 37-42, which is incorporated by reference in its entirety. By performing Fourier transform infrared (FTIR) spectroscopy on the aliquots of reaction solutions at different times during the heating process (FIGS. 2A-2H), it was found that the characteristic absorption peaks of the vinyl group (═C—H bend (909.7 and 991.2 cm⁻¹); C═C stretch (1641.4 cm⁻¹)) decreased during reflux at 320° C. for 1 hour (FIG. 2B), consistent with vinyl group oxidation. This observation was in line with previous reports demonstrating that alkene oxidation can reduce selenium dioxide into metallic selenium (see, for example, Chen, O.; Chen, X.; Yang, Y.; Lynch, J.; Wu, H.; Zhuang, J.; Cao, Y. C. Angewandte Chemie International Edition 2008, 47, (45), 8638-8641, which is incorporated by reference in its entirety) and Fe³⁺ into metallic iron. See, for example, Shavel, A.; Liz-Marzan, L. M. Physical Chemistry Chemical Physics 2009, 11, (19), 3762-3766, which is incorporated by reference in its entirety. By contrast, DBE undergoes free radical decomposition into toluene and benzaldehyde during thermolysis (FIG. 2C). See, for example, Gilbert, K. E.; Gajewski, J. J. The Journal of Organic Chemistry 1982, 47, (25), 4899-4902, which is incorporated by reference in its entirety. New peaks associated with the characteristic vibrational frequencies of an aromatic aldehyde (C═O stretch (1700 cm⁻¹); C═O bend (827 cm⁻¹)) (see, for example, Baxendale, J. H.; Magee, J. Discuss. Faraday Soc. 1953, 14, 160-169, which is incorporated by reference in its entirety) emerged during synthesis indicating formation of benzaldehyde. To confirm free-radical generation during DBE thermolysis reactions were performed in mixtures of co-solvents containing of 4:1, 2:1, and 1:1 ODE to DBE volume ratios. Progressively higher oxidation of the vinyl group in ODE was observed with increasing proportions of DBE (FIG. 2D, FIG. 8). The integrated absorbance of the ═C—H band at ˜910 cm⁻¹ after 30 minutes of reflux was normalized to the initial value at 200° C. Elimination of up to ˜60% (˜13 mmol) of the vinyl groups was observed in 1:1 ODE:DBE mixture compared to just ˜4% (˜2 mmol) oxidized in pure ODE.

Solvent Optimized Redox Tuning Yields Nearly Defect-Free MNPs.

While ferrite MNPs with dimensions <10 nm are appropriate for applications in MR imaging, magnetic hyperthermia demands larger nanoparticles for efficient heat dissipation. According to dynamic hysteresis modeling, the loss power is maximized for ferrite MNPs at the superparamagnetic to ferromagnetic transition for low alternating magnetic field amplitudes, and in the ferromagnetic regime for higher amplitudes. This transition corresponds to iron oxide MNPs 20-30 nm in size that are approximately spherical for alternating magnetic field frequencies f on the order of hundreds of kilohertz. See, for example, Christiansen, M. G.; Senko, A. W.; Chen, R.; Romero, G.; Anikeeva, P. Applied Physics Letters 2014, 104, (21), -, which is incorporated by reference in its entirety. To fully decompose FeOl₃ and grow the MNPs to this size range, reaction temperatures exceeding 300° C. must be attained. See, for example, Bronstein, L. M.; Huang, X.; Retrum, J.; Schmucker, A.; Pink, M.; Stein, B. D.; Dragnea, B. Chemistry of Materials 2007, 19, (15), 3624-3632, which is incorporated by reference in its entirety. Pure DBE provides an in-situ oxidation mechanism that prevents wüstite formation, but its low reflux temperature of 290° C. sets an upper limit of ˜7 nm on MNP dimensions in the conditions tested. Solvent optimized redox tuning (SORT) was relied on to access temperatures >300° C. while selectively promoting the formation of the inverse spinel phase. It was found that ODE:DBE solvent mixtures with volume ratios of 4:1 and 2:1 had reflux temperatures of 330° C. and 325° C. respectively. Additionally, changing the molar ratio of oleic acid to FeOl₃ in these co-solvent systems provided a convenient means to tune the MNP dimensions (FIG. 2E). An ODE:DBE mixture with a volume ratio of 1:1 exhibited a reflux temperature of 310° C., which was too low to synthesize nanoparticles >15 nm. Monodisperse (σ<10%) faceted ferrite MNPs with sizes of 10±0.8 nm, 15±1.5 nm, 19±1.8 nm, and 27±1.9 nm were produced using oleic acid to FeOl₃ ratios of 1:1, 3:2, 2:1, and 3:1 respectively in an ODE:DBE solvent mixture with a 2:1 volume ratio (FIG. 2F-2H). Faceted FiM 26±2.2 nm nanoparticles were also synthesized with a 2:1 volumetric ratio mixture of SQE:DBE (T=330° C.) and a molar ratio of oleic acid to FeOl₃ of 2:1. Without the addition of DBE, only biphasic FiM/AFM nanoparticles could be synthesized. Note that the direct addition of the DBE decomposition product benzaldehyde to ODE did not assist in the oxidation process (FIG. 9).

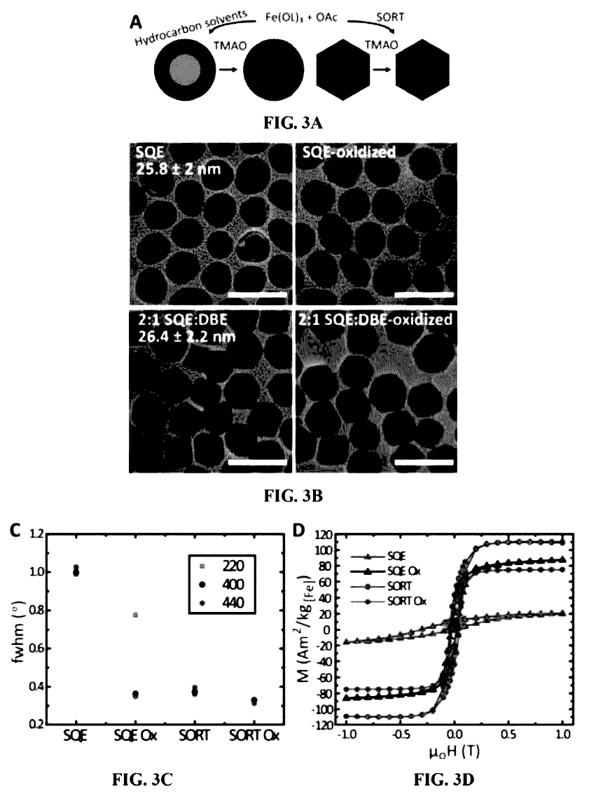

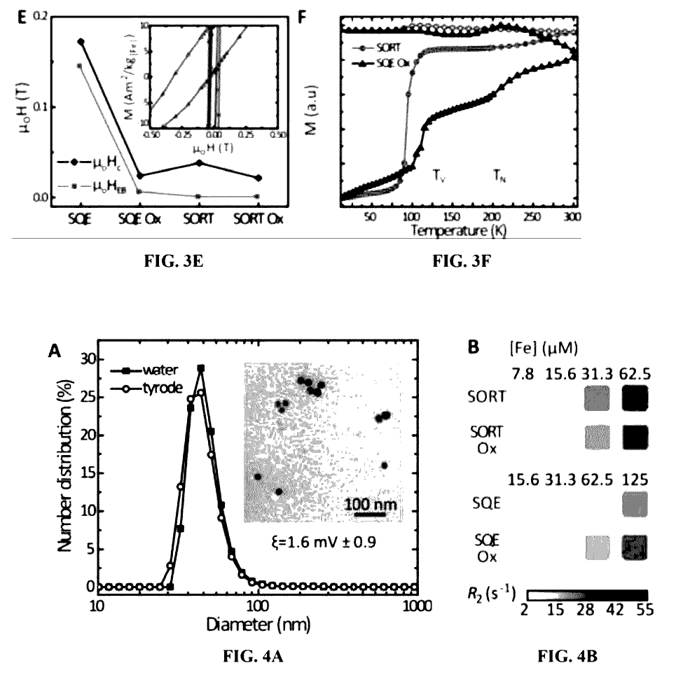

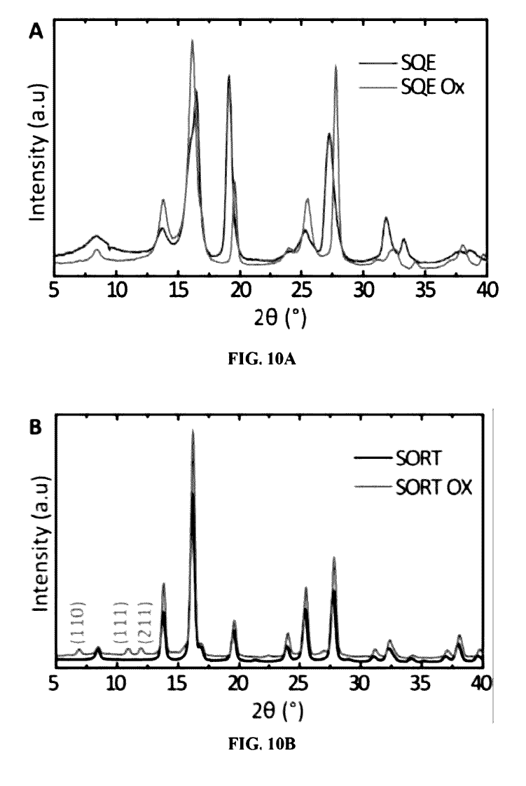

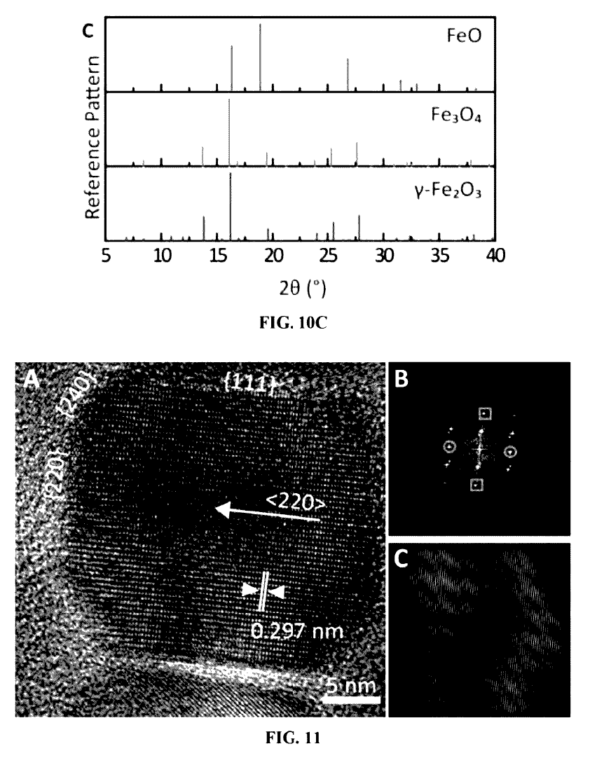

The impact of defects was then investigated on the structural and magnetic properties of ferrite MNPs by comparing ˜25 nm nanoparticles synthesized by SORT (co-solvent volume of 2:1 SQE:DBE) with those produced in pure SQE (FIG. 3A-3F). SQE was a convenient solvent choice due to its higher boiling point compared to ODE to synthesize similarly-sized nanoparticles >20 nm for the pure and co-solvent conditions tested. To assess the effects of iron valency, a post-synthesis oxidation procedure was applied to both types of particles (FIGS. 3A and 3B). XRD patterns revealed that oxidation with trimethyl amine N-oxide (TMAO) for 30 minutes at 140° C. was sufficient to convert biphasic MNPs synthesized in SQE into inverse spinel Fe_{3-δ}O₄ (FIG. 10A). Application of the same oxidation treatment to SORT nanoparticles led to the conversion of Fe_{3-δ}O₄ into γ-Fe₂O₃, detected as small increases (˜0.2°) of higher angle peaks and the emergence of weak diffraction peaks corresponding to the (110), (111), and (211) planes (FIG. 10B). HRTEM of SORT nanoparticles further confirmed that only the inverse spinel phase was synthesized (FIG. 11). To evaluate the impact of the redox processes on crystal structure, the full width at half-maximum (fwhm) values for the (220), (400), and (440) peaks associated with the inverse spinel phase were determined from line profile fits to XRD patterns for nanoparticles produced in SQE and co-solvents (SORT) prior to and following post-synthesis oxidation (FIG. 3C). For biphasic nanoparticles synthesized in SQE, it was observed fwhm values of ˜1° for all three diffraction planes with significant reduction in line broadening for the (400) and (440) peaks following oxidation, while the (220) line was not affected to the same degree. Because both the octahedrally-coordinated iron interstitials and oxygen sublattice of the inverse spinel (400)/(440) and rock salt (200)/(220) diffraction peaks overlap, defects along these crystallographic directions can contribute to anisotropic strain broadening as observed in the biphasic nanoparticles. See, for example, Wetterskog, E.; Tai, C.-W.; Grins, J.; Bergström, L.; Salazar-Alvarez, G. ACS Nano 2013, 7, (8), 7132-7144; Mittemeijer, E. J.; Welzel, U. Zeitschrift für Kristallographie International journal for structural, physical, and chemical aspects of crystalline materials 2008, 223, (9), 552-560, each of which is incorporated by reference in its entirety. Tetrahedrally coordinated iron in the inverse spinel solely contributes to the diffraction line along the (220) direction, which should not be significantly influenced by oxidation treatment. Fwhm <0.4° was measured for all planes for MNPs produced by SORT independent of post-synthesis oxidation, suggestive of the low defect density within these nanoparticles.

Field-cooled magnetization curves were collected at 5 K to identify differences in magnetic properties between the nanoparticles produced from the four synthetic protocols depicted in FIG. 3B. Characteristic of coupled systems with FiM and AFM subdomains, exchange bias (H_{EB}) and coercivity (H_{c}) was present in the hysteresis loop of biphasic MNPs synthesized in SQE (FIGS. 3D and 3E). Although oxidation treatment increased the Mₛ from ˜20 to 87 A·m²/kg_{[Fe]}, residual H_{EB} of ˜60 mT and high-field susceptibility persisted in these materials due to the presence of antiphase boundaries and other structural defects that could not be eliminated likely due to insufficient cation diffusion during oxidation. By contrast, it was found that ferrite nanoparticles synthesized in co-solvent mixtures exhibited Mₛ (110 A·m²/kg_{[Fe]}) values approaching those of bulk magnetite (128 A·m²/kg_{[Fe]}) and H_{EB}=O (FIGS. 3D and 3E). Post-synthesis oxidation of nanoparticles produced by SORT into γ-Fe₂O₃ resulted in a decrease in Mₛ to 75 A·m²/kg_{[Fe]}. Temperature-dependent magnetization curves of SORT-synthesized MNPs exhibited a sharp increase in magnetization near its Verwey temperature (T_{V}=123 K), characteristic of F_{e3-δ}O₄ as it undergoes a metal-to-insulator transition (FIG. 3F). See, for example, Lee, J.; Kwon, S. G.; Park, J.-G.; Hyeon, T. Nano Letters 2015, 15, (7), 4337-4342, which is incorporated by reference in its entirety. Oxidized Fe_{3-δ}O₄ nanoparticles synthesized in pure SQE exhibited a suppressed Verwey transition, indicative of potential short-range cation disorder impeding this charge-ordering phenomenon. See, for example, Wetterskog, E.; Tai, C.-W.; Grins, J.; Bergström, L.; Salazar-Alvarez, G. ACS Nano 2013, 7, (8), 7132-7144; Hai, H. T.; Yang, H. T.; Kura, H.; Hasegawa, D.; Ogata, Y.; Takahashi, M.; Ogawa, T. Journal of Colloid and Interface Science 2010, 346, (1), 37-42; Senn, M. S.; Wright, J. P.; Attfield, J. P. Nature 2012, 481, (7380), 173-176, each of which is incorporated by reference in its entirety. Furthermore, an increase in magnetization above the Neél temperature (T_{N}=192 K) of wüstite suggests that the AFM phase cannot be completely eliminated by oxidation.

Elimination of Defects Enhances the Transverse Relaxivity and Specific Loss Power of Nanoparticles.

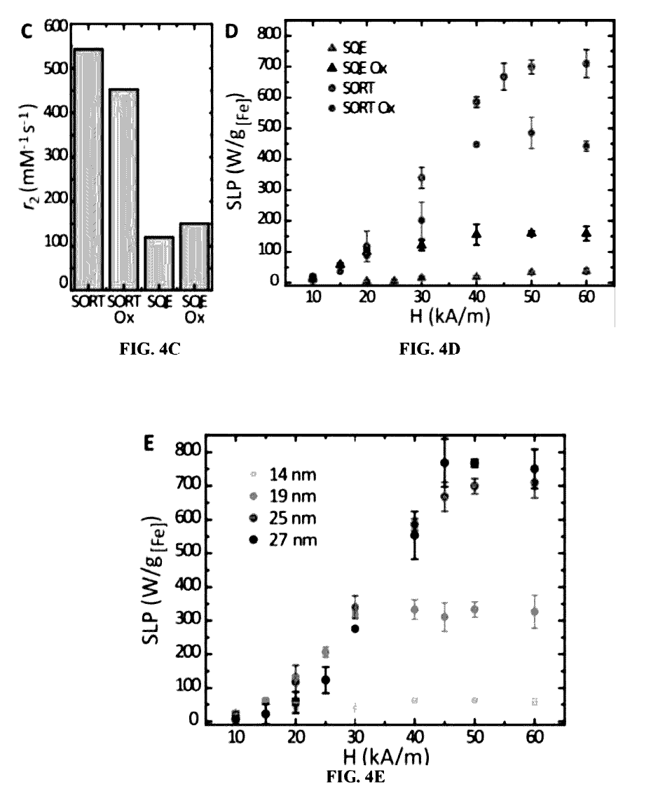

The MR relaxivity and hyperthermic performance for the MNPs presented in FIGS. 3A-3F were compared for their potential application as theranostic agents. A room temperature ligand exchange protocol was adopted to intercalate amphiphilic poly(maleic anhydride-alt-1-octadecene) modified with poly(ethylene glycol) (PEG) (PMAO-PEG) with the surface oleates to render the nanoparticles water-soluble. See, for example, Yu, W. W.; Chang, E.; Falkner, J. C.; Zhang, J.; Al-Somali, A. M.; Sayes, C. M.; Johns, J.; Drezek, R.; Colvin, V. L. Journal of the American Chemical Society 2007, 129, (10), 2871-2879, which is incorporated by reference in its entirety. 25 nm nanoparticles passivated with PMAO-PEG were colloidally stable at physiological conditions with a charge neutral surface (FIG. 4A). The transverse relaxivity r₂ values of the PEG-MNPs synthesized by SORT was measured and compared them to similarly-sized particles prepared in SQE and subjected to oxidation (FIGS. 4B and 4C). SORT-synthesized MNPs exhibited an r₂ of 543 mM⁻¹s⁻¹, which was 3.6 times greater than the r₂ determined for oxidized MNPs containing defects. Note that r₂ values of 740 mM⁻¹s⁻¹ near the theoretical limit of 800 mM⁻¹s⁻¹ were previously observed for 28 nm iron oxide nanocubes synthesized in pure DBE, which similarly required phase-pure ferrimagnetic nanoparticles dispersed as single core magnets to observe these exceptionally high values. See, for example, Lee, N.; Choi, Y.; Lee, Y.; Park, M.; Moon, W. K.; Choi, S. H.; Hyeon, T. Nano Letters 2012, 12, (6), 3127-3131, which is incorporated by reference in its entirety.

Hyperthermic performance, quantified as specific loss power (SLP), was assessed by exposing the MNP solutions to an alternating magnetic field (AMF) and recording the temperature change as a function of time. SLPs for ˜25 nm nanoparticles presented in FIGS. 3A-3F are depicted in FIG. 4D, at different AMF amplitudes (Hₒ=10-60 kA/m) and a frequency off=100 kHz. Biphasic 25 nm MNPs (SQE) exhibited negligible SLP values (<50 W/g_{[Fe]} at 100 kHz and 50 kA/m) that increased by a factor of 4 to 160 W/g_{[Fe]} following oxidation (SQE Ox) (FIG. 4D). Notably at the same AMF conditions, 25 nm SORT nanoparticles had SLP values of 700 W/g_{[Fe]}. The 4.7 fold improvement highlights the necessity in preventing defect formation during the reaction. While SQE nanoparticles exhibit improved magnetic properties following oxidation, residual defects may serve as pinning layers to prevent full coherent reversal of the magnetic moment needed to maximize hysteretic losses. See, for example, Arora, S. K.; Sofin, R. G. S.; Nolan, A.; Shvets, I. V. Journal of Magnetism and Magnetic Materials 2005, 286, 463-467, which is incorporated by reference in its entirety. As a result, the measured SLP is lower than predicted. For SORT-synthesized nanoparticles, size-dependent increase of the SLP with increasing AMF amplitude was observed that reached a plateau at ˜40 kA/m (FIG. 4E), which is qualitatively consistent with trends predicted by the dynamic hysteresis model. See, for example, Christiansen, M. G.; Senko, A. W.; Chen, R.; Romero, G.; Anikeeva, P. Applied Physics Letters 2014, 104, (21), -, which is incorporated by reference in its entirety. SLP values of 750 W/g_{[Fe]} at 45 kA/m and 100 kHz were measured for 27 nm MNPs synthesized via SORT. This value is amongst the highest recorded for AMF conditions within the clinical limit (<Hₒ·f=5×10⁹ A·m⁻¹·s⁻¹). See, for example, Guardia, P.; Riedinger, A.; Nitti, S.; Pugliese, G.; Marras, S.; Genovese, A.; Materia, M. E.; Lefevre, C.; Manna, L.; Pellegrino, T. Journal of Materials Chemistry B 2014, 2, (28), 4426-4434; Young, J. H.; Wang, M. T.; Brezovich, I. A. Electronics Letters 1980, 16, (10), 358-359, each of which is incorporated by reference in its entirety. Notably, it was observed at an AMF with an amplitude and frequency product <50% of the fields previously used to demonstrate comparable SLP values in ferrite MNPs. See, for example, Guardia, P.; Di Corato, R.; Lartigue, L.; Wilhelm, C.; Espinosa, A.; Garcia-Hernandez, M.; Gazeau, F.; Manna, L.; Pellegrino, T. ACS Nano 2012, 6, (4), 3080-3091; Chen, R.; Christiansen, M. G.; Anikeeva, P. ACS Nano 2013, 7, (10), 8990-9000, each of which is incorporated by reference in its entirety. These findings illustrate that defect elimination during the synthesis yields nanomagnetic agents with enhanced theranostic performance.

Improved Hyperthermic Performance Enables Rapid Wireless Magnetothermal Control of Intracellular Calcium.

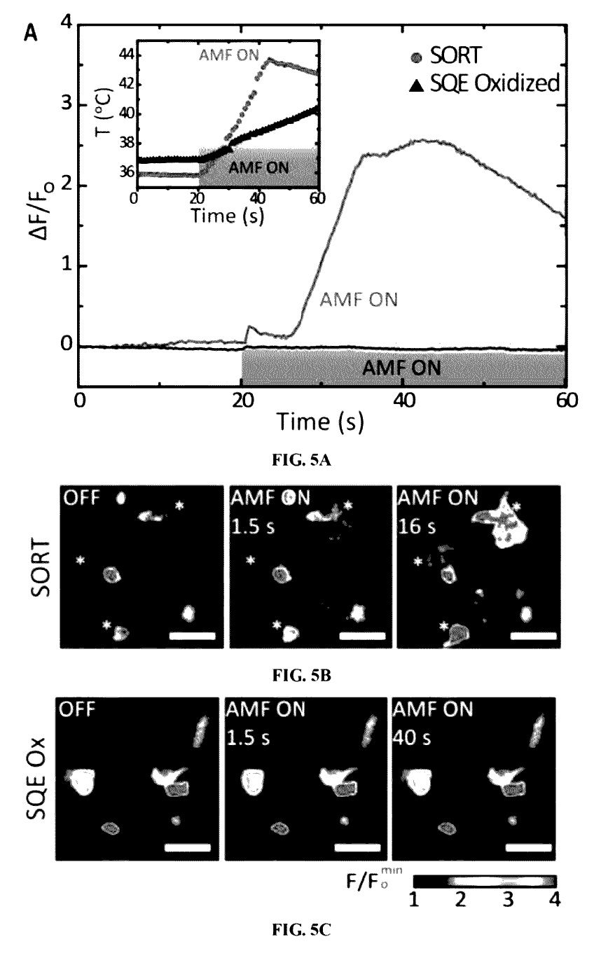

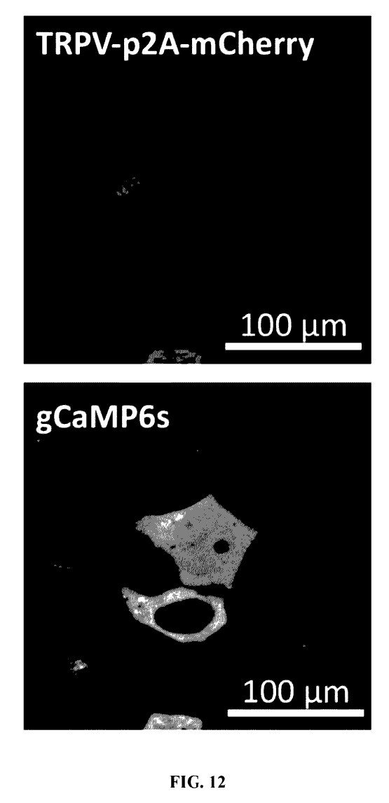

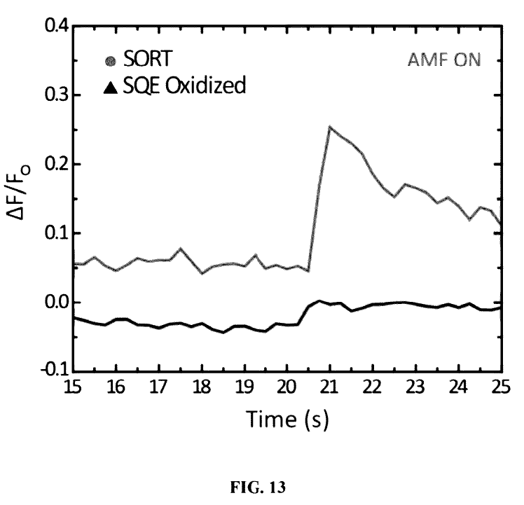

Single-crystalline Fe_{3-δ}O₄ nanoparticles with high SLPs can act as efficient transducers to convert AMF into a rapid thermally mediated calcium ion (Ca²⁺) influx in heat-sensitized cells, which is desirable for applications in wireless neural excitation and control of gene transcription. See, for example, Chen, R.; Romero, G.; Christiansen, M. G.; Mohr, A.; Anikeeva, P. Science 2015, 347, (6229), 1477-1480; Stanley, S. A.; Gagner, J. E.; Damanpour, S.; Yoshida, M.; Dordick, J. S.; Friedman, J. M. Science 2012, 336, (6081), 604-608, each of which is incorporated by reference in its entirety. Human embryonic kidney cells (HEK293FT) were co-transfected using lipofectamine with a heat-sensitive Ca²⁺ channel TRPV1 and the genetically encoded calcium indicator gCaMP6s for dynamic quantification of intracellular calcium concentration changes by fluorescence microscopy (FIG. 12). See, for example, Chen, R.; Romero, G.; Christiansen, M. G.; Mohr, A.; Anikeeva, P. Science 2015, 347, (6229), 1477-1480, which is incorporated by reference in its entirety. The HEK293FT cells were immersed in solutions of MNPs (2 mg/mL of [Fe]) dispersed in Tyrode's solution, a physiological buffer. AMF (f=150 kHz, Hₒ=32 kA/m) was applied externally using a home-built inductive coil driven by a series resonant circuit as previously described. See, for example, Chen, R.; Romero, G.; Christiansen, M. G.; Mohr, A.; Anikeeva, P. Science 2015, 347, (6229), 1477-1480, which is incorporated by reference in its entirety. Within 20 s of an applied AMF, temperature of the SORT-synthesized MNP solution (SLP=770±50 W/g_{[Fe]}) increased from 36° C. to 43° C., surpassing the thermal threshold for TRPV1 activation. The AMF-induced Ca²⁺-influx was manifested as a fluorescence change (ΔF/Fₒ>50%) in 75% of the 120 cells counted across 3 trials (FIGS. 5A and 5B). Notably, HEK293FT cells surrounded by SORT-synthesized MNPs exhibited minor and major activation onsets with latencies of ˜750 ms and ˜5 s respectively (FIG. 13). While the latter influx of Ca²⁺ is due to the bulk temperature increase within the ferrofluid, the former may potentially be attributed to the local heat dissipation by MNPs adjacent to the cell membranes. Consistent with poor heat dissipation, a solution of oxidized MNPs synthesized in SQE at the same particle concentration was not able to reach 40° C. even after 40 s of AMF exposure, resulting in insignificant intracellular Ca²⁺ increase (FIGS. 5A and 5C). The improved temporal control over intracellular Ca²⁺ levels with SORT-synthesized nanoparticles is directly correlated to its efficient heat dissipation, which may be therapeutically advantageous in lowering the MNP concentration and AMF exposure time required for other biomedical applications such as cancer hyperthermia.

### CONCLUSION

Redox engineering with nonaqueous solution chemistry was demonstrated to bias a ferrite nanoparticle reaction into its thermodynamically favored FiM configuration. A co-solvent strategy enabled synthesis of nearly defect-free Fe_{3-δ}O₄ nanoparticles through in-situ generation of oxidizing radicals while reaching temperatures exceeding 320° C. to promote growth to particle sizes in the superparamagnetic to ferromagnetic transition regime. Structural and magnetic characterization of MNPs synthesized by SORT revealed low anisotropic peak broadening, lack of exchange bias and high-field susceptibility, Mₛ near bulk values, and a pronounced Verwey transition. These hallmark features of nearly defect-free magnetite nanoparticles are absent in nanoscale ferrites prepared by conventional oxidation methods. Structural optimization of the MNPs led to their enhanced performance as MR contrast agents with r₂ values exceeding 500 mM⁻¹s⁻¹, and as magnetothermal transducers with SLP values of 750 W/g_{[Fe]} recorded for 27 nm nanoparticles at clinically relevant AMF conditions. The latter translated into wireless control of intracellular Ca²⁺ concentration with sub-second latencies. Adjusting the electrochemical potential of the solvent environment is a facile strategy to tune the phase composition within magnetic ferrites and may be extended to other transition metal oxides requiring fine control over the redox state of the material.

Relevant abbreviations follow:

Acac, acetylacetonate; AFM, antiferromagnetic; AMF, alternating magnetic field; DBE, dibenzyl ether; FiM, ferrimagnetic; HRTEM, high-resolution transmission electron microscopy; MNP, magnetic nanoparticle; MR, magnetic resonance; Mₛ, saturation magnetization; ODE, 1-octadecene; Ol, oleate; SQE, squalene; SORT, solvent optimized redox transformation; TMAO, trimethyl amine N-oxide; XRD, x-ray diffraction.

### Examples

Materials and Methods.

Sodium oleate (95%, TCI America) and iron chloride hexahydrate (99%+, Acros) were purchased from different vendors. All other solvents and reagents were purchased from Sigma-Aldrich: oleic acid (90%), octadecane (99%), 1-octadecene (90%), squalene (99%), dibenzyl ether (98%), dioctyl ether (99%), trimethylamine N-oxide (98%), poly(maleic anhydride-alt-1-octadecene) (Mₙ=30,000-50,000), and poly(ethylene glycol) methyl ether (Mₙ=5000).

Synthesis of Metal-Oleate Complex.

In a 1 L 3 neck flask, 30 mmol of FeCl₃.6H₂O and 92 mmol of sodium oleate was heated to reflux (60° C.) in a solvent mixture comprised of 200 mL of hexane, 100 mL of ethanol, and 100 mL of ddH₂O for one hour under N₂. The hexane layer containing the iron-oleate complex was then extracted with a separatory funnel and washed twice with ddH₂O. The iron-oleate mixture was heated to 110° C. in a beaker and dried overnight stirring on a hotplate.

Synthesis of Magnetic Nanoparticles with Different Solvents.

In a 250 mL 3 neck flask, 5 mmol of iron-oleate, 2.5 mmol of oleic acid, and 20 mL of solvent (octadecane, 1-octadecene, squalene, dioctyl ether, or dibenzyl ether) was degassed at 90° C. for 30 minutes. Then the mixture was heated to 200° C. under N₂, then to reflux at 3.3° C./min and held at the reflux temperature for 30 minutes. The nanoparticles were extracted by transferring the reaction solution into 50 mL conical tubes, adding 30 mL of ethanol to promote flocculation, then precipitated by centrifuging at 10,000 rpm for 10 minutes. Following two washes (disperse in hexane followed by the addition of ethanol then centrifugation), the nanoparticle pellet was re-dispersed in 10 mL of chloroform.

Synthesis of Magnetic Nanoparticles Using SORT.

In a 250 mL 3 neck flask, 5 mmol of iron-oleate and 5 mmol (10 nm), 10 mmol (15 nm), 12.5 mmol (19 nm), or 15 mmol (27 nm) of oleic acid was combined in a 2:1 volume ratio of 1-octadecene (10 mL) and dibenzyl ether (5 mL) and degassed at 90° C. for 30 minutes. Then the mixture was heated to 200° C. under N₂, then to reflux (325° C.) at 3.3° C./min and held at the reflux temperature for 30 minutes. After pelleting and washing, the nanoparticle pellet was re-dispersed in 5 mL of chloroform.

Oxidation of as-Synthesized Nanoparticles.

After cooling the reaction solution to room temperature, 15 mmol of trimethylamine N-oxide was added, heated to 140° C. in air, and allowed to react for 30 minutes.

FTIR.

Aliquoted reactions collected at different time points were diluted 1:10 in chloroform. 10 μL of this solution was drop-casted then sandwiched between NaCl windows (International Crystal Laboratories). FTIR spectra was collected on a Thermo Fisher FTIR6700 Spectrometer using transmission mode. Aliquots during the course of a reaction were drawn using a 1 mL gas-tight Hamilton syringe.

Structural and Magnetic Characterization.

Powder x-ray diffraction patterns of as-synthesized nanoparticles was collected on a three-circle diffractometer coupled to a Bruker-AXS Smart Apex charged-coupled-device (CCD) detector with graphite-monochromated Mo K a radiation (λ=0.71073 Å). Field-cooled (5T) hysteresis curves at 5 K were measured using a superconducting quantum interference device (SQUID, MPMS-XL, Quantum Design). SQUID temperature dependent magnetization curves were measured with an applied field of 10 mT. Room temperature hysteresis curves were generated on a vibrating sample magnetometer (VSM, Digital Measurement Systems Model 880A).

Phase transfer and PEGylation. 100 μL of nanoparticle solution dispersed in chloroform (˜10 mg/mL) was combined with 1 mL of poly(ethylene glycol) grafted poly(maleic anhydride-alt-1-octadecene) solution (10 mg/mL in chloroform) and sonicated for 15 minutes. After evaporating the chloroform under vacuum, 2 mL of 1×Tris/Borate/EDTA buffer was added and sonicated for 30 minutes. The nanoparticles were magnetic separated and washed twice with water, then reconstituted in 1 mL of water (˜1 mg/mL) and sonicated for 10 minutes.

Elemental Analysis.

Nanoparticles were digested in 37% v/v HCl overnight at 60° C. and diluted in 2 wt % HNO₃. Inductively coupled plasmon emission spectroscopy (ICP-ES, Jobin-Yvon Ultima-C) was used to quantify the elemental concentration.

SLP Measurements.

PEG-coated MNPs were adjusted to 2 mg/mL prior to SLP measurements. A custom-built series resonant circuit powered by a 200 W amplifier (1020L, Electronics & Innovation) was used to generate alternating AMF, with the field amplitude measured with a pickup coil and oscilloscope. Temperature measurements were made with a fiber optic temperature probe (Omega HHTFO-101).

MR Imaging.

Mill experiments were performed on a 7 T PharmaScan® MRI instrument (Bruker). The relaxivity of the samples were determined by using the MSME (multi-slice multi-echo) sequence at room temperature with the following: TR (repetition time)=2 s, 30 echoes with 24 ms TE (echo time) averaged over 4 acquisitions, FOV (field of view)=5×5 cm, matrix=256×256, and section thickness=2 mm.

HEK293FT Cell Experiments.

HEK293FT cells were seeded on 5 mm cover glass coated with matrigel and transfected with TRPV1-p2A-mCherry and gCaMP6s using lipofectamine. Cells were placed in a 7.5 mm gap cut into a soft ferromagnetic core and immersed in 1.5 mg/mL [Fe] of nanoparticles. An AMF off=150 kHz and Hₒ=30 kA/m was applied while real-time fluorescence recordings of gCaMP6s was captured on an inverted microscope as previously described. See, for example, Guardia, P.; Riedinger, A.; Nitti, S.; Pugliese, G.; Marras, S.; Genovese, A.; Materia, M. E.; Lefevre, C.; Manna, L.; Pellegrino, T. Journal of Materials Chemistry B 2014, 2, (28), 4426-4434, which is incorporated by reference in its entirety.

Other embodiments are within the scope of the following claims.

What is claimed is:

1. A method of preparing a redox-active nanoparticle comprising:
selecting a solvent to optimize redox tuning of a reaction medium;
decomposing a precursor compound at a reflux temperature of the solvent to produce the nanoparticle.

2. The method of claim 1, further comprising oxidizing the nanoparticle.

3. The method of claim 2, wherein the oxidized nanoparticle is an inverse spinel phase iron oxide.

4. The method of claim 1, wherein the oxidized nanoparticle includes iron, manganese, cobalt, nickel, copper, or zinc or binary or ternary mixtures thereof.

5. The method of claim 1, wherein the solvent includes dibenzyl ether, diphenyl ether, anisole, phenetole, an aromatic ester, an aromatic acetal, an aromatic aminal or an aromatic anhydride.

6. The method of claim 1, wherein the solvent is a mixture.

7. The method of claim 6, wherein the mixture includes a high boiling point alkene and a high boiling point ether.

8. The method of claim 7, wherein the high boiling point is at least 280° C.

9. The method of claim 7, wherein the solvent includes a mixture of two or more of octadecane, 1-octadecene (ODE), squalene (SQE), dioctyl ether, and dibenzyl ether (DBE).

10. The method of claim 1, wherein the compound includes an iron oleate.

11. The method of claim 1, wherein the nanoparticle includes ferrite.

12. The method of claim 1, wherein the nanoparticle has a size of between 7 and 30 nm.

13. A magnetic nanoparticle population comprising a plurality monodisperse ferrite particles having a size of between 7 and 30 nm.

14. A method of imaging comprising
introducing the nanoparticle population of claim 13 into a subject; and
creating a magnetic particle imaging signal of the subject.
